SEQUENCE LISTING (19 sequences)

SEQ ID NO: 1 - DNA, 20 bp, Artificial Sequence
  Description of Artificial Sequence Synthetic oligonucleotide
tccatgacgttcctgacgtt

SEQ ID NO: 2 - DNA, 18 bp, Artificial Sequence
  Description of Artificial Sequence Synthetic oligonucleotide
tctcccagcgtgcgccat

SEQ ID NO: 3 - DNA, 22 bp, Artificial Sequence
  Description of Artificial Sequence Synthetic oligonucleotide
tcgtcgttttcggcgcgcgccg

SEQ ID NO: 4 - DNA, 20 bp, Artificial Sequence
  Description of Artificial Sequence Synthetic oligonucleotide
tccatgacgttcctgatgct

SEQ ID NO: 5 - DNA, 24 bp, Artificial Sequence
  Description of Artificial Sequence Synthetic oligonucleotide
tcgtcgttttgtcgttttgtcgtt

SEQ ID NO: 6 - DNA, 21 bp, Artificial Sequence
  Description of Artificial Sequence Synthetic oligonucleotide
tccaatgagcttcctgagtct

SEQ ID NO: 7 - DNA, 20 bp, Artificial Sequence
  Description of Artificial Sequence Synthetic oligonucleotide
tccaggacttctctcaggtt

SEQ ID NO: 8 - DNA, 20 bp, Artificial Sequence
  Description of Artificial Sequence Synthetic oligonucleotide
tccatgaggttcctgatgct

SEQ ID NO: 9 - DNA, 22 bp, Artificial Sequence
  Description of Artificial Sequence Synthetic oligonucleotide
tcgtcgttttccggcgcgccgg

SEQ ID NO: 10 - DNA, 22 bp, Artificial Sequence
  Description of Artificial Sequence Synthetic oligonucleotide
tcgtcgttttcggcggccgccg

SEQ ID NO: 11 - DNA, 12 bp, Artificial Sequence
  Description of Artificial Sequence Synthetic oligonucleotide
tcgagcgttctc

SEQ ID NO: 12 - DNA, 20 bp, Artificial Sequence
  Description of Artificial Sequence Synthetic oligonucleotide
ggtgcatcgatgcagggggg

SEQ ID NO: 13 - DNA, 23 bp, Artificial Sequence
  Description of Artificial Sequence Synthetic oligonucleotide
tcgtcgttttgtcgttttctcgt

SEQ ID NO: 14 - protein, 448 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 20 bp, Artificial Sequence
  Description of Artificial Sequence Synthetic oligonucleotide
ggggtcaacgttgagggggg

SEQ ID NO: 17 - DNA, 20 bp, Artificial Sequence
  Description of Artificial Sequence Synthetic oligonucleotide
gggggacgatcgtcgggggg

SEQ ID NO: 18 - DNA, 22 bp, Artificial Sequence
  Description of Artificial Sequence Synthetic oligonucleotide
tcgtcgttttcggcgcgcgccg

SEQ ID NO: 19 - DNA, 19 bp, Artificial Sequence
  Description of Artificial Sequence Synthetic oligonucleotide
gggggacgatcgtcggggg

(19) United States
(12) Patent Application Publication
(10) Pub. No.: US 2023/0331849 A1
(43) Pub. Date: Oct. 19, 2023

(54) CpG IMMUNOCONJUGATES FOR CANCER THERAPY

(71) Applicant: University of Southern California, Los Angeles, CA (US)

(72) Inventors: Alan Epstein, Los Angeles, CA (US); Peisheng Hu, Los Angeles, CA (US); Leslie Khawli, Los Angeles, CA (US)

(21) Appl. No.: 18/044,274

(22) PCT Filed: Sep. 8, 2021
(86) PCT No.: PCT/US2021/049532

§ 371 Date: Mar. 7, 2023

Related U.S. Application Data
(60) Provisional application No. 63/076,014, filed on Sep. 9, 2020.

(51) Int. Cl. C07K 16/28; A61K 47/68; A61K 31/4406

(52) U.S. Cl. CPC C07K 16/2827; A61K 47/6849; A61K 47/6807; A61K 31/4406

(57) ABSTRACT

Applicant discloses herein an immunoconjugate, which enables systemic use of targeted CpG using a new class of antibodies which target the tumor microenvironment. Conjugation to checkpoint inhibitors such as anti-PD-L1, anti-CTLA-4, anti-LAGS anti-TIM-3, and anti-VISTA to make “biobetter” reagents.

### CROSS-REFERENCE TO RELATED APPLICATION

This application is a 371 of International Patent Application No. PCT/US2021/049532, filed Sep. 8, 2021, which in turn claims priority under 35 U.S.C. § 119(e) of U.S. Provisional Application No. 63/076,014, filed Sep. 9, 2020, the contents of each are hereby incorporated by reference into this application in their entireties.

### SEQUENCE LISTING

The instant application contains a Sequence Listing which has been submitted electronically in XML format and is hereby incorporated by reference in its entirety. Said XML copy, created on Mar. 2, 2023, is named 064189-1093.xml and is 26,433 bytes in size.

### BACKGROUND

There is a general need in the art for cancer therapeutics that are effective and offer the potential to counteract the ability of cancer cells to evade the immune system. Efforts to develop such therapeutics have further been hampered by the inability to find effective therapeutics that can be administered systemically rather than locally.

### SUMMARY

In the last several years, the implementation of checkpoint inhibitor antibodies into clinical practice for the treatment of solid tumors has spurred the interest of oncologists to identify other treatments that can work synergistically with this new class of therapeutics. From these studies, it has been shown that innate immune agonists such as TLR-4 (oncolytic viruses), the Sting pathway, and Toll Like Receptor 9 (TLR-9) (CpG) are perhaps the most potent enhancers of checkpoint inhibition associated with the PD-1/PD-L1 pathway. As a non-limiting example only, Applicant shows herein that an antibody or antigen binding fragment thereof directed to PD-L1 conjugated to CpG produces a biobetter checkpoint inhibitor. PD-L1 has the added advantage of being expressed on the surface of tumor cells as well as antigen presenting cells, the very cells that harbor the receptors for TLR-9. Without being bound by theory, conjugation of CpG with αPD-L1 alters the toxicity profile of the checkpoint inhibitor in a favorable manner making thereby improving its use in the clinic.

Provided herein is an immunoconjugate, the immunoconjugate comprising, or alternatively consisting essentially of, or yet further consisting of, an immune checkpoint inhibitor linked to an oligonucleotide comprising, or alternatively consisting essentially of, an immunostimulatory sequence motif which contains at least one unmethylated CG dinucleotide. In one aspect, the immunoconjugate also comprises, or consists essentially of, or yet further consists of a detectable and/or a purification label. In one aspect, the immune checkpoint inhibitor is an antibody or an antigen binding fragment thereof, comprising, or alternatively consisting essentially of the CDRs of the immune checkpoint inhibitor. Non-limiting examples of such include an anti-PD-L1 antibody, an anti-CTLA-4 antibody, an anti-LAG3 antibody, an anti-TIM-3 antibody, or an anti-VISTA antibody or antigen binding fragments thereof.

In one aspect, the immunostimulatory sequence motif is CpG oligodeoxynucleotide (ODN), optionally a CpG Class-A ODN, a CpG Class-B ODN, or a CpG Class-C ODN. In a further aspect, the immunostimulatory sequence is TLR 9 agonist CpG that is optionally thiol-modified and, further optionally comprises CpG1826 or CpG 2006. In one aspect, the ODN comprises the sequence GTCCTT or GACFTT. In one aspect, the immune checkpoint inhibitor is linked covalently, optionally via a cross-linker, to the immunostimulatory sequence motif. In a further aspect is it linked to primary amines on the antibody or fragment thereof. In a further aspect, the crosslinker is an amine-to-sulfhydryl crosslinker, optionally Sulfo-EMCS. Further provided are polynucleotides encoding the polypeptide backbone of the immunoconjugate and the ODN, vectors and host cells containing them.

Further provided are compositions comprising, or alternatively consisting essentially of, or yet further consisting of, one or more of the immunoconjugate and a carrier such as a pharmaceutically acceptable carrier.

The compositions are intended for use in cancer therapy to reduce the size, tumor burden or metastatic potential of the tumor. Thus, provided herein is a method of treating a tumor or cancer comprising, or alternatively consisting essentially of, or yet further consisting of, administering an effective amount of: the immunoconjugate, the isolated polynucleotide, the vector, or the isolated host cell, and/or the composition to a subject in need thereof. In one aspect, the cancer cells or tumors express an immune checkpoint. In another aspect they express the immune checkpoint and are resistant to immune checkpoint therapy. The subject can be an animal in need of therapy, such as a canine, a feline, or a human patient. The therapy can be combined with another anti-tumor or anti-cancer therapy by administering an effective amount of an anti-tumor or anti-cancer therapy, examples of which are provided herein. They can be administered concurrently or subsequent or serially. The immunoconjugate can be administered as a first line, a second line, a third line, or a fourth line therapy. In one aspect, the immunoconjugate is administered subsequent to cytoreductive therapy.

Also provided herein is a method to inhibit the growth of a cancer cell or a tumor cell. In one aspect the cancer cell or tumor expresses an immune checkpoint. In another aspect the cancer or tumor cell expresses the immune checkpoint and is resistant to immune checkpoint therapy. The method comprises or consists essentially of, or yet further consists of contacting the cancer or tumor cell with an effective amount of: the immunoconjugate, the isolated polynucleotide, the vector, or the isolated host cell, and/or the composition to a subject in need thereof. The cancer cell or tumor cells can be an animal such as a mammal, e.g., a canine cell, a feline cell, or a human cell. The cell or tumor can be contacted with another anti-tumor or anti-cancer therapy. The contacting can be in vitro or in vivo. When contacted in vitro, the method provides a screen or assay for new therapies or for pre-screening personalized therapy. When contacted in vivo in a non-human animal, the method provides an animal model to test new therapies.

Kits are further provided herein. The kit can comprise any one or more of the immunoconjugate, the isolated polynucleotide, the vector, the isolated host cell, and/or the composition as described herein and instructions for use. In one aspect, the instructions are for use in treating a cancer and/or tumor.

Also provided herein are compositions and methods for making the immunoconjugate both through recombinant expression and/or chemical cross-linking.

### BRIEF DESCRIPTION OF THE DRAWINGS

FIG. 1 shows the structure of 3 classes of CpG (InvivoGen). Figure discloses SEQ ID NOS 19, 5 and 3, respectively, in order of appearance.

FIGS. 2A-2B show the effect of chTNT-3/CpG 1826 on the growth of B16 melanoma (FIG. 2A) and Colon 26 murine tumor models (FIG. 2B) compared to direct injection of free CpG1826 and antibody treatment alone.

FIGS. 3A-3B show the results of in vivo therapy studies of combination versus conjugated CpG with anti-PD-L1 (Tecentriq, Roche) antibody in D2F2 bearing murine breast carcinoma tumors. In FIG. 3A, therapies were administered by i.p. injections q.d. X5 as indicated by the black arrows. In FIG. 3B, tumor growth ia plot of the geometric mean RTV. (Mean±SEM is plotted. Representative of 1 experiment (n=5/group). AIC therapy significance relative to indicated treatment group based on unpaired t-test analysis. *P<0.05, ***P<0.001. Note, two groups of mice [CpG+αPD-L1 and αPD-L1] showed marked scabbing of the tumor surface requiring early termination of these mice.

FIG. 4 shows that in a second model of murine breast cancer, mice bearing the metastatic triple negative 4T1 tumor model showed a 50% survival rate when treated with the AIC (CpG1826/anti-PD-L1) compared to anti-PD-L1 treatment alone (n=10). When the HDAC inhibitor, Entinostat, was used in combination with the AIC, the survival rate increased to 60%. Consistent with results seen in the clinic in patients, breast cancer is relatively resistant to anti-PD-L1 checkpoint blockade and in this experiment, only 10% of the PD-L1 treated control group survived by 26 days. These results demonstrate that systemic targeting of CpG to the tumor microenvironment can significantly improve the immunotherapy of highly resistant tumors such as breast cancer.

FIG. 5 shows Tecentriq chemically linked to the Toll-Like receptor 9 agonist CpG inhibited the tumor volume of treated mice.

### DETAILED DESCRIPTION

### Definitions

Throughout and within this disclosure various technical and patent literature are referenced with a bibliographic citation or a reference to a citation that may be found immediately preceding the claims. The disclosures of the technical and patent literature are hereby incorporated by reference into the present disclosure in their entireties.

Unless defined otherwise, all technical and scientific terms used herein have the same meanings as commonly understood by one of ordinary skill in the art to which this disclosure pertains. All nucleotide sequences provided herein are presented in the 5′ to 3′ direction. Although any methods and materials similar or equivalent to those described herein can be used in the practice or testing of the present disclosure, the preferred methods, devices, and materials are now described. All technical and patent publications cited herein are incorporated herein by reference in their entireties. Nothing herein is to be construed as an admission that the invention is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (TRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

As used in the specification and claims, the singular form “a”, “an” and “the” include plural references unless the context clearly dictates otherwise. For example, the term “a polypeptide” includes a plurality of polypeptides, including mixtures thereof.

The term “about,” as used herein when referring to a measurable value such as an amount or concentration and the like, is meant to encompass variations of 20%, 10%, 5%, 1%, 0.5%, or even 0.1% of the specified amount.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of disclosed herein. Embodiments defined by each of these transition terms are within the scope of this invention.

As used herein, the term “immunoconjugate” refers to an antibody linked to a second molecule.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

The term “protein,” “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The terms “equivalent” or “biological equivalent” are used interchangeably when referring to a particular molecule, biological, or cellular material and intend those having minimal homology while still maintaining desired structure or functionality.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, “linked” means that under physiological conditions of pH, ionic strength and osmotic potential, the majority of the entities are associated with each other at equilibrium. Covalent linkage may be by any of a variety of chemical linking and crosslinking agents including, for example, homobifunctional or heterobifunctional crosslinking reagents, many of which are commercially available (see, e.g., Pierce Chemical Co. or Sigma Chemical Co.). Linking or crosslinking can be achieved by any of a variety of chemistries well known in the art including, for example, activated polyethylene glycols, aldehydes, isocyanates, maleimides and the like.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

In certain embodiments, the terms “disease” “disorder” and “condition” are used interchangeably herein, referring to a cancer, a status of being diagnosed with a cancer, or a status of being suspect of having a cancer.

“Cancer” which is also referred to herein as “tumor” is a known medically as an uncontrolled division of abnormal cells in a part of the body, benign or malignant. As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, breast cancer, melanoma, ovarian cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas. In one aspect, the cancer cell or tumor expresses an immune checkpoint. In a further aspect, the cancer or tumor expresses an immune checkpoint and is resistant to checkpoint inhibitor therapy. As used herein, the term “resistant to checkpoint inhibitor therapy” intends that the cell expresses the immune checkpoint but growth or metastasis of the cell or tumor is not delayed such that clinical use of the checkpoint inhibitor is warranted.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises breast cancer, colon cancer, bladder cancer, bone cancer, ovarian cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, glioma, cervical cancer, hepatocellular, thyroid cancer, or stomach cancer.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

As used herein, the terms “treating,” “treatment,” and the like are used herein to mean obtaining a desired pharmacologic and/or physiologic effect. The effect may be prophylactic in terms of completely or partially preventing a disease, disorder, or condition or sign or symptom thereof, and/or may be therapeutic in terms of a partial or complete cure for a disorder and/or adverse effect attributable to the disorder. In one aspect, treatment is a reduction in tumor burden, a reduction in tumor size, remission or an inhibition of metastatic potential or metastasis of the tumor. In aspect, the term excludes a prophylactic effect or prevention of cancer.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

B16 melanoma is a murine tumor cell line used as a model for human skin cancers. They also are useful to study metastasis and solid tumor formation. See Overwijk and Restifo, Curr. Protoc Immunol. (2001) May; Chapter Unit-20, doi:10.1002/0471142735.im2001s39. The cells are commercially available from the American Type Culture Collection (ATCC).

Colon 26 is murine tumor model cell line and is commercially available from the ATCC and are resistant to checkpoint inhibitor therapy. See Kim K, Skora A D, Li Z, et al. Eradication of metastatic mouse cancers resistant to immune checkpoint blockade by suppression of myeloid-derived cells. Proc Natl. Acad Sci USA 2014; 11 (32):11774-11779. doi:10.1073/pnas.141062611.

D2F2 is a murine mammary tumor cell line that is HER2-negative.

Murine breast cancer carcinoma 4T1 is a transplantable tumor cell line that is highly tumorigenic and invasive. It can spontaneously metastasize from the primary tumor in the mammary gland to multiple distal sites including lymph nodes, blood, liver, lung, brain and blood. See Pulaski and Ostrand-Rosenberg (2001) Curr. Protoc. Immunol. May; Chapter Unit-20, doi:10.1002/0471142735.im2001s39. The cells are commercially available from the ATCC. It is a triple-negative mouse breast cancer cell linen and resistant to checkpoint inhibitors. See Kim K, Skora A D, Li Z, et al. Eradication of metastatic mouse cancers resistant to immune checkpoint blockade by suppression of myeloid-derived cells. Proc Natl Acad Sci USA. 2014; 111(32):11774-11779. doi:10.1073/pnas.1410626111.

As used herein, an “immune checkpoint” or “checkpoint” refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

A checkpoint inhibitor is type of drug that blocks proteins called checkpoints that are made by some types of immune system cells, such as T cells, and some cancer cells. These checkpoints help keep immune responses from being too strong and sometimes can keep T cells from killing cancer cells. When these checkpoints are blocked, T cells can kill cancer cells better. Examples of checkpoint proteins found on T cells or cancer cells include PD-1/PD-L1 and CTLA-4/B7-1/B7-2. Some immune checkpoint inhibitors are used to treat cancer and examples of such are provided herein.

In some embodiments, the checkpoint inhibitor comprises, consists essentially of, or consists of a peptide that includes a protein or a fragment thereof from one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, B1754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or R07121661. In some embodiments, the anti-TIGIT agent comprises, consists essentially of, or consists of MK-7684, etigilimab, tiragolumab, BMS-986207, AB-154, or ASP-8374. In some embodiments, the anti-VISTA agent comprises, consists essentially of, or consists of JNJ-61610588 or CA-170. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004. In some embodiments, the anti-Siglec-15 agent comprises, consists essentially of, or consists of NC318. In some embodiments, the checkpoint inhibitor comprises, consists essentially of, or consists of AK104, KN046 Tecentiriq® (atezolizumab). Atezolizumab is a monoclonal antibody used to treat a variety of tumors including triple-negative breast cancer, small cell lung cancer, hepatocellular carcinoma, non-small cell lung cancer and urothelial carcinoma. It is a fully humanized monoclonal antibody of IgGI isotype against PD-L1. A published sequence of the antibody is: (Heavy chain) EVQLVESGGG LVQPGGSLRL SCAASGFTFS DSWIHWVRQA PGKGLEWVAW ISPYGGSTYY ADSVKGRFTI SADTSKNTAY LQMNSLRAED TAVYYCARRH WPGGFDYWGQ GTLVTVSSAS TKGPSVFPLA PSSKSTSGGT AALGCLVKDY FPEPVTVSWN SGALTSGVHT FPAVLQSSGL YSLSSVVTVP SSSLGTQTYI CNVNHKPSNT KVDKKVEPKS CDKTHTCPPC PAPELLGGPS VFLFPPKPKD TLMISRTPEV TCVVVDVSHE DPEVKFNWYV DGVEVHNAKT KPREEQYAST YRVVSVLTVL HQDWLNGKEY KCKVSNKALP APIEKTISKA KGQPREPQVY TLPPSREEMT KNQVSLTCLV KGFYPSDIAV EWESNGQPEN NYKTTPPVLD SDGSFFLYSK LTVDKSRWQQ GNVFSCSVMH EALHNHYTQK SLSLSPGK (SEQ ID NO: 14) and (Light chain) DIQMTQSPSS LSASVGDRVT ITCRASQDVS TAVAWYQQKP GKAPKLLIYS ASFLYSGVPS RFSGSGSGTD FTLTISSLQP EDFATYYCQQ YLYHPATFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC (SEQ ID NO: 15), available at https://www.genome.jp/entry/D10773, last accessed on Sep. 7, 2021.

In some embodiments, the anti-PD1 agent comprises, consists essentially of, or consists of an anti-PD1 antibody or an antigen binding fragment thereof. In some embodiments, the anti-PD1 antibody comprises, consists essentially of, or consists of nivolumab, pembrolizumab, cemiplimab, spartalizumab, camrelizumab, sintilimab, tislelizumab, toripalimab, AMF 514 (MEDI0680), balstilimab, or a combination of two or more thereof.

In some embodiments, the anti-PD-L1 agent or fragment thereof comprises, consists essentially of, or consists of an anti-PD-L1 antibody or an antigen binding fragment thereof. In some embodiments, the anti-PD-L1 antibody comprises, consists essentially of, or consists of avelumab, durvalumab, atezolizumab, envafolimab, or a combination of two or more thereof.

In some embodiments, the checkpoint inhibitor comprises, consists essentially of, or consists of a peptide or fragment thereof of an anti-PD1 agent or an anti-PD-L1 agent. In one aspect, the anti-PD-L1 antibody is atezolizumab or an antigen binding fragment thereof.

In some embodiments, the checkpoint inhibitor comprises, consists essentially of, or consists of an anti-CTLA-4 agent or an antigen binding fragment thereof. In some embodiments, the anti-CTLA-4 agent comprises, consists essentially of, or consists of an anti-CTLA-4 antibody or an antigen binding fragment thereof. In some embodiments, the anti-CTLA-4 antibody comprises, consists essentially of, or consists of ipilimumab, tremelimumab, zalifrelimab, or AGEN1181, an antigen binding fragment thereof or a combination thereof.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

In some aspects, an additional therapy is contacted or administered to the subject. These can include adjuvant or neoadjuvant treatments, e.g., a HDAC inhibitor such as Entinostat. The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

Alternatively or additionally, the additional agents can include therapies to minimize side effects or to enhance the therapy or treatment such as chemotherapeutic agents, non-limiting example of such are provided herein. In one aspect, an additional therapy comprises 5-Fluorouracil (5-FU) which belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

A further example is “5-FU based adjuvant therapy” that refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine (methyl-CCNU), vincrisine (Oncovin®) and 5-FU). For a review of these therapies see Beaven and Goldberg (2006) Oncology 20(5):461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Another example is capecitabine which is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is another example. It is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[[(2-amino-5-formyl-1,4,5,6,7,8-hexahydro-4-oxo-6-pteridinyl)methyl]amino]benzoyl], calcium salt (1:1).

Another example is “oxaliplatin” (Eloxatin) which is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

A further example of an additional therapy is “FOLFOX” which is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

A further example includes Irinotecan (CPT-11) which is sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

“Cytoreductive therapy,” as used herein, refers to cancer therapy aimed at debulking a cancerous tumor. Such therapy includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Toposiomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m2 to not more than about 50 J/m2, usually about 10 J/m2.

A further example is a histone deacetylase inhibitor (HDAC) inhibitor such as Entinostat. Other non-limiting examples include Trichostatin A (TSA), Vorinostat (SAHA), Panobinostat (LBH589, NVP-LBH589), 4-Phenylbutyric acid, Valproic acid, KA2507, Pomiferin, see https://www.medchemexpress.com/Targets/HDAC.html?locale=es-ES?src=googleproduct&gclid=EAIaIQobChMI76nR7fPv8gIVTD6tBh2pJAjdEAAYASABE gKhxfD_BwE, last accessed Sep. 8, 2021. Histone deacetylase inhibitors exert their anti-tumor effects via the induction of expression changes of oncogenes or tumour suppressor, through modulating the acetylation/deactylation of histones and/or non-histone proteins such as transcription factors.

Entinostat is also known as SNDX-275 and MS-275 is a benzamide histone deacetylase inhibitor. The preferred IUPAC name is (Pyridin-3-yl)methyl ({4-[(2-aminophenyl)carbamoyl]phenyl}methyl)carbamate and has the chemical formula C₂₁H₂₀N₄O₃. It is commercially available from AdooQ Bioscience (https://www.adooq.com/ms-275-entinostat.html, last accessed on Sep. 8, 2021) and Focus Biomolecules (https://focusbiomolecules.com/ms-275-entinostat-hdac-inhibitor/, last accessed on Sep. 8, 2021).

Yet further examples include biologics such as monoclonal antibodies and therapies derived from such.

In some embodiments, the terms “first” “second” “third” “fourth” or similar in a component name are used to distinguish and identify more than one components sharing certain identity in their names. For example, “first cell line” and “second cell line” are used to distinguishing two cell lines.

A “gene product” or alternatively a “gene expression product” refers to the amino acid (e.g., peptide or polypeptide) generated when a gene is transcribed and translated. In some embodiments, the gene product may refers to an mRNA generated when a gene is transcribed.

“Under transcriptional control”, which is also used herein as “directing expression of”, is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “a regulatory sequence” “an expression control element” or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EF1alpha promoter and the CMV promoter. The EF1alpha sequence is known in the art (see, e.g., addgene.org/11154/sequences/; ncbi.nlm.nih.gov/nuccore/J04617, each last accessed on Mar. 13, 2019, and Zheng and Baum (2014) Int'l. J. Med. Sci. 11(5):404-408). The CMV promoter sequence is known in the art (see, e.g., snapgene.com/resources/plasmid-files/?set=basic_cloning_vectors&plasmid=CMV_promoter, last accessed on Mar. 13, 2019 and Zheng and Baum (2014), supra.).

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The polypeptide or equivalents of each thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

An equivalent thereof comprises an polypeptide having at least 80% amino acid identity to a reference polypeptide that is encoded by a polynucleotide that hybridizes under conditions of high stringency to the complement of a polynucleotide encoding the polypeptide, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In one aspect, the antibody or antigen binding fragment thereof is defined by one or more CDRs. In one embodiment, it includes one, or two or all three of the CDRs (e.g., CDR1, CDR2, CDR3) from the LC variable region with appropriate CDRs from other antibody CDRs, and equivalents of each thereof. Accordingly, and as an example, the CDR1 and CDR2 from the LC variable region can be combined with the CDR3 of another antibody's LC variable region, and in some aspects, can include an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In one aspect all 6 CDRs from one antibody are utilized in the antibody or antigen binding fragment thereof.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

The term “antibody variant” intends to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this disclosure. Derivatives include, but are not limited to, for example, bispecific, multispecific, heterospecific, trispecific, tetraspecific, multispecific antibodies, diabodies, chimeric, recombinant and humanized.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹ M, or 10⁻¹² M.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated. Non-limiting examples of route of administration include oral administration, nasal administration, injection, and topical application.

Administration or treatment in “combination” refers to administering two agents such that their pharmacological effects are manifest at the same time. Combination does not require administration at the same time or substantially the same time, although combination can include such administrations.

“Simultaneous use” as used herein refers to the administration of the two compounds of the composition according to the invention in a single and identical pharmaceutical form or at the same time in two distinct pharmaceutical forms.

“Separate use” as used herein refers to the administration, at the same time, of the two compounds of the composition according to the invention in distinct pharmaceutical forms.

“Sequential use” as used herein refers to the successive administration of the two compounds of the composition according to the invention, each in a distinct pharmaceutical form.

“Pharmaceutically acceptable carriers” refers to any diluents, excipients, or carriers that may be used in the compositions of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of disease or stage of disease, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M−1 greater, at least 104 M−1 greater or at least 105 M−1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a 3-sheet conformation and the CDRs form loops which connect, and in some cases form part of, the 3-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds PD-L1 will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, a fragment crystallizable (Fc) region refers to the tail region of an antibody that in some embodiments, can serve to stabilize the antibody and optionally interacts with (such as binds) an Fc receptor on an immune cell or on a platelet or that binds a complement protein.

When used in this context the prefix “anti-” and the descriptor “antibody” refer to an antibody, fragment, derivative, or biological equivalent thereof that recognizes or binds the recited protein, e.g., anti-PD-L1 antibody recognizes and binds PD-L1.

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as an N-terminal or C-terminal fragment the target polypeptide or an isolated polypeptide. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of antibodies having different amino acid sequences. In one aspect, the polyclonal antibody comprises a mixture of plural types of antibodies having different CDRs. As such, a mixture of cells which produce different antibodies is cultured, and an antibody purified from the resulting culture can be used (see WO 2004/061104).

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

A host cell can be a eukaryotic or a prokaryotic cell. “Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification label” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

Administration or treatment in “combination” refers to administering two agents such that their pharmacological effects are manifest at the same time. Combination does not require administration at the same time or substantially the same time, although combination can include such administrations.

“Immunotherapy,” as used herein, refers to cancer therapies that enhance the immune response to a tumor or cancer. Such therapy includes but is not limited to adoptive cell therapies, such as those utilizing CAR T-cells, CD4+ or CD8+ cytotoxic cells, natural killer cells, or equivalents thereof; monoclonal antibodies and immunoconjugate based therapies designed to target and destroy tumors and/or cancer cells; cytokine therapy, such as interferon gamma (“IFNγ”) treatment; and vaccination.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov. Typically, a patient is given a subsequent chemotherapy regimen because the patient did not show a positive clinical or sub-clinical response to the first line therapy or the first line therapy has stopped.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

CpG oligodeoxynucleotides (ODNs) are synthetic oligonucleotides that are comprised of unmethylated CpG dinucleotides which are arranged in a specific sequence of framework known as CpG motifs (Gursel et al. J Leukoc Biol 71: 813-820 (2002)). CpG motifs trigger the production of T-helper 1 and pro-inflammatory cytokines and stimulate the activation of professional antigen-presenting cells (APCs) which include macrophages and dendritic cells. Unmethylated CpG ODNs behave as immune adjuvants, accelerating and enhancing antigen-specific antibody responses. CpG ODNs interact with Toll-like receptor (TLR) 9 to trigger the maturation and functional activation of professional antigen presenting cells, B-cells and natural killer cells (Ballas, Immunol. Res. 39: 15-21 (2007); Jakob et al. Int. Arch. Allergy Immunol. 118: 457-461 (1999); Krieg et al. Annu. Rev. Immunol. 20:709-760 (2002); Krug et al. Eur. J. Immunol. 31: 2154-2163 (2001); Zhu et al. J. Biol. Chem. 284: 22878-22887 (2009)). They are quickly internalized by immune cells, through a speculated pathway involving phophatidylinositol 3-kinases (PI3Ks), and interact with TLR9 present in cytoplasmic endosomes. The resultant immune response is characterized by the production of polyreactive immunoglobulin (IgM) antibodies, cytokines, and chemokines directed towards the induction of T-helper 1 immunity. The TLR9 receptor recognizes CpG ODNs with a strict bias for the chemical and conformational nature of the unmethylated CpG ODN. The conjugation of an oligonucleotide and a CpG DNA at the 5′-end has been shown to reduce significantly the immunostimulatory activity of the CpG DNA. By contrast, the conjugation of an oligonucleotide and a CpG DNA at the 3′-end has an insignificant effect on the immunostimulatory activity (Li et al. J Immunol Methods 389:45-51 (2013); Jang et al. Cancer Immunol Immunother, 65(5):511-23 (2016)). In fact, it has been shown that the immunostimulatory activity of the CpG DNA is enhanced by 3′ sequence modifications.

Non-limiting examples of CpG ODN include CPG are CpG 1826 and CpG 2006. CpG 1826 comprises, or consists essentially of, or yet further consists of the sequence 5′-TCC ATG ACG TTC CTG ACG TT-3′ (SEQ ID NO: 1) (commercially available from InvivoGen, https://www.invivogen.com/odn, last accessed on Sep. 8, 2021), and optionally wherein all bases of the CpG ODN have the phosphorothioate backbone and further optionally wherein the last base is substituted with a 3-′thiomodifier C3 and yet further optionally, the modified CpG is linked to the primary amines on the antibody or an antigen binding fragment thereof. In a further aspect, the CpG 1826 comprises, or consists essentially of, or yet further consists of the sequence 5′-TCC ATG ACG TTC CTG ACG TT-3′ (SEQ ID NO: 1), and wherein all bases of the CpG 1826 have the phosphorothioate backbone and wherein the last base is substituted with a 3-′thiomodifier C3 and optionally, the modified CpG is linked to the primary amines on the antibody or an antigen binding fragment thereof. CpG 2006 comprises, or consists essentially of, or yet further consists of the sequence 5′-TCG TCG TTT TGT CGT TTT GTC GTT-3′ (SEQ ID NO: 5) (commercially available from Invivogen, https://www.invivogen.com/sites/default/files/invivogen/products/files/odn2006_t ds.pdf, last accessed on Sep. 8, 2021), and optionally wherein all bases of the CpG ODN have the phosphorothioate backbone and further optionally wherein the last base is substituted with a 3-′thiomodifier C3 and yet further optionally, the modified CpG is linked to the primary amines on the antibody or an antigen binding fragment thereof. In a further aspect, the CpG 1826 comprises, or consists essentially of, or yet further consists of the sequence 5′-TCG TCG TTT TGT CGT TTT GTC GTT-3′ (SEQ ID NO: 5), and wherein all bases of the CpG 2006 have the phosphorothioate backbone and wherein the last base is substituted with a 3-′thiomodifier C3 and optionally, the modified CpG is linked to the primary amines on the antibody or an antigen binding fragment thereof.

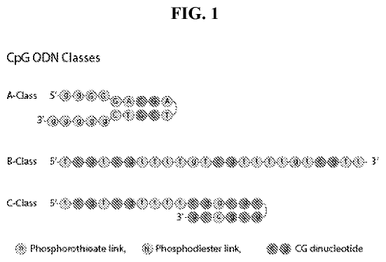

It has been established that at least three classes of CpG ODNs exist: CpG-A (Type A), CpG-B (Type B), and CpG-C (Type C) (Krieg. Nature Rev Drug Discovery, 5:471-484 (2006)). CpG-A ODN has been identified as being a potent inducer of natural killer cell activation and interferon-a secretion, whereas CpG-B ODN elicits predominant B-cell proliferation. More recently a Type C class represented by SD-101 (Dynavax) is being tested in the clinic. A summary of the structure (FIG. 1) and function (Table 1) of each class is shown below.

One area of cancer immunotherapy in which CpG DNA has shown surprising efficacy in mouse models is as a monotherapy (Kim et al. Immunology, 112: 117-125 (2004)). It appears that direct injection of CpG ODN into tumor lesions activates local dendritic cells and induces the production of IL-12 in and around the tumor. In several different tumor models, injection of CpG-B ODN has led to regression of established tumors in a T-cell dependent fashion (Corrales et al. Cell Rep. 11(7):1018-1030 (2015)). In a B-16 melanoma model, injection of CpG-A ODNs either into the tumor or systemically has led to tumor regression in an NK dependent, T-cell independent manner.

A second use of CpG ODN in tumor immunotherapy is in combination with antitumor antibodies (Duraiswamy et al. Cancer Research, 73(23), 6900-6912; Sagiy-Barfi et al. Sci Transl Med. 10:eaan4488, (2018); Gallotta et al. Cancer Research, canres.0729.2018 (2018)). Administration of CpG ODN dramatically activates ADCC effector cells and induces expression of CD64. When this is followed by injection of an antitumor antibody, dramatic increases in biologic activity are seen. Regression can be achieved with large tumors that would not normally respond to antibody therapy, as well as with tumors that only express the target antigen at a low concentration.

TABLE 1
Definition of CpG classes (InvivoGen)

CpG-A ODNs are characterized by a PO central CpG-containing
palindromic motif and a PS-modified 3′ poly-G string. They induce high
IFN-α production from pDCs but are weak stimulators of TLR9-
dependent NF-κB signaling and pro-inflammatory cytokine (e.g. IL-6)
production.
CpG-B ODNs contain a full PS backbone with one or more CpG
dinucleotides. They strongly activate B cells and TLR9-dependent NF-κB
signaling but weakly stimulate IFN-α secretion.
CpG-C ODNs combine features of both classes A and B. They contain a
complete PS backbone and a CpG-containing palindromic motif. C-Class
CpG ODNs induce strong IFN-α production from pDC as well as B cell
stimulation

### Modes for Carrying Out the Disclosure

This disclosure provides an immunoconjugate, the immunoconjugate comprising, or alternatively consisting essentially of, or yet further consisting of, an immune checkpoint inhibitor linked to an oligonucleotide, the oligonucleotide comprising, or alternatively consisting essentially of, or yet further consisting of, an immunostimulatory sequence motif which contains and is linked to at least one unmethylated CG dinucleotide. In one aspect, the immunoconjugate also comprise a detectable and/or purification label.

In one aspect, the immune checkpoint inhibitor is an antibody or an antigen binding fragment thereof. Non-limiting examples of such are provided herein, e.g., an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, BI754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or R07121661, or an antigen binding fragment thereof. In some embodiments, the anti-TIGIT agent comprises, consists essentially of, or consists of MK-7684, etigilimab, tiragolumab, BMS-986207, AB-154, or ASP-8374 or an antigen binding fragment thereof. In some embodiments, the anti-VISTA agent comprises, consists essentially of, or consists of JNJ-61610588 or CA-170 or an antigen binding fragment thereof. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004 or an antigen binding fragment thereof. In some embodiments, the anti-Siglec-15 agent comprises, consists essentially of, or consists of NC318 or an antigen binding fragment thereof. In some embodiments, the checkpoint inhibitor comprises, consists essentially of, or consists of AK104 or KN046 or an antigen binding fragment thereof.

In one aspect, the immune checkpoint inhibitor is of the group of an anti-PD-L1 antibody, an anti-CTLA-4 antibody, an anti-LAG3 antibody, an anti-TIM-3 antibody, or an anti-VISTA antibody or an antigen binding fragment thereof. In one aspect the checkpoint inhibitor an anti-PD-L1 antibody or antigen binding fragment thereof such as atezolizumab.

In another aspect, the immunostimulatory sequence motif is CpG oligodeoxynucleotide (ODN), optionally a CpG Class-A ODN, a CpG Class-B ODN, or a CpG Class-C ODN. In a further aspect, all bases of the CpG ODN have the phosphorothioate backbone and the last base is substituted with a 3-′thiomodifier C3 and optionally, the modified CpG is linked to the primary amines on the antibody or fragment thereof. In a further aspect, the CpG comprises CpG 1826 e.g. comprising 5′-TCC ATG ACG TTC CTG ACG TT-3′ (SEQ ID NO: 1) and all bases of the CpG ODN have the phosphorothioate backbone and the last base is substituted with a 3-′thiomodifier C3 and optionally, the modified CpG is linked to the primary amines on the antibody or fragment thereof. In another aspect, the CpG comprises CpG 2006 and optionally, the modified CpG is linked to the primary amines on the antibody or fragment thereof. See https://www.idtdna.com/pages/education/decoded/article/synthetic-cpg-odns-activate-immune-cells-through-the-toll-like-receptor-(tlr)-pathway, last accessed on Sep. 7, 2021 and Bauer et al. (2001) PNAS, Vol. 98(16):9237-9242.

As used herein, “linked” means that under physiological conditions of pH, ionic strength and osmotic potential, the majority of the entities are associated with each other at equilibrium. Covalent linkage may be by any of a variety of chemical linking and crosslinking agents including, for example, homobifunctional or heterobifunctional crosslinking reagents, many of which are commercially available (see, e.g., Pierce Chemical Co. or Sigma Chemical Co.). Linking or crosslinking can be achieved by any of a variety of chemistries well known in the art including, for example, activated polyethylene glycols, aldehydes, isocyanates, maleimides and the like.

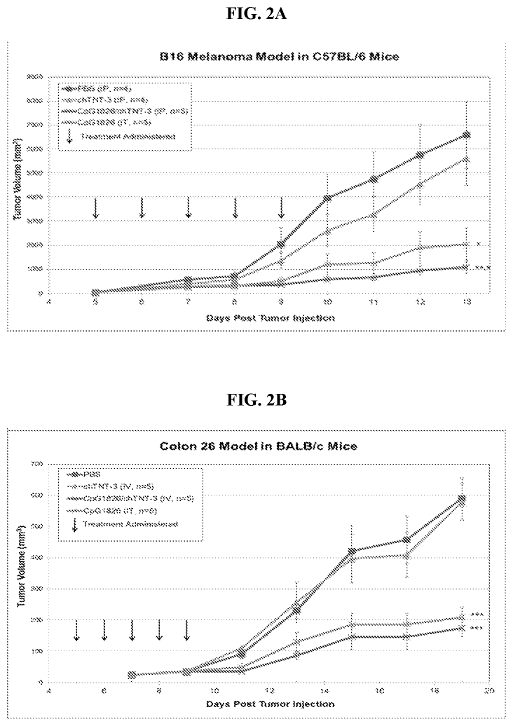

The immunoconjugates attach CpG motifs to antibodies that target tumors as a clinically relevant reagent for cancer immunotherapy. To accomplish this, Applicants established methods to chemically link modified CpG motifs to antibodies using bifunctional linkers. Such methods are known in the art, for example as published in Li et al. J Immunol Methods 389:45-51 (2013)). As detailed below, several tumor models have been tested with the immunoconjugates as described herein, including the murine B16 melanoma, colon 26 tumors, a triple negative tumor and PD-L1 resistant breast cancer. When used as a monotherapy, the chTNT-3/CpG immunoconjugate significantly regressed the fast growing murine tumors to a similar extent as mice treated with direct intratumoral CpG injection unlike chTNT-3 controls (FIGS. 2A-2B). These results demonstrate that CpG can be effectively conjugated to antibodies and can induce tumor regression by engaging innate immune defenses.

The antibody portion of the immunoconjugate can be derived from any source and can be selected on binding affinity and/or avidity. The antibody portion or antigen binding fragment thereof must retain the ability to bind to the tumor antigen. Non-limiting examples of antigens include antigens associated with tumors, e.g., oncofetal antigens, oncoviral antigens, overexpressed or accumulated antigens, cancer testis antigens, CT10 or CT10 antigens, lineage restricted antigens, mutated antigens, postranslationally altered antigens, or idiotypic antigens. See, e.g. Zarour et al. “Categories of Tumor Antigens” in Holland-Frei Cancer Medicine. 6th edition. Hamilton (ON): BC Decker (2003). More specifically, the antigen is an antigen associated with an immune checkpoint molecule, e.g., PD-L1, CTLA-4, LAG3, TIM-3, and VISTA, and, therefore, its antibody serves as an immune checkpoint inhibitor. The antibodies and fragments thereof can be commercially available or prepared using conventional techniques. The antibodies can be polyclonal, monoclonal, and from any appropriate species, murine, bovine, canine, equine or human.

As noted above, the term “immune checkpoint” refers to molecules that prevent the immune system from attacking cells indiscriminately. An “immune checkpoint inhibitor” is a molecule that inhibits the immune checkpoint molecule. An example of such is PD-L1.

“PD-1” or “programmed cell death protein 1” or “CD279” or “cluster of differentiation 279” is a protein that is encoded by the PD-1 gene and is a cell surface receptor belonging to the immunoglobulin (Ig) superfamily that plays a role in down-regulating immune functions, promoting self-tolerance, suppressing T-cell inflammatory activity, suppressing apoptosis by antigen specific T-cells in the lymph nodes, and reducing apoptosis of T-regs. PD-1 binds PD-L1 and PD-L2. Due to its immune regulatory function, PD-1 is often referred to as an immune checkpoint. Non-limiting exemplary amino acid sequences for PD-1 can be found in the Uniprot database under accession numbers Q15116 (human PD-1), Q02242 (murine PD-1), U6CTF8 (mink PD-1); other homologs of the same may also be found in the Uniprot database, i.e., at www.uniprot.org. Detection of a cell expressing PD-1 can be identified using conventional techniques, such as the use of an anti-PD-1 antibody, which are commercially available, e.g., from a vendor such as Abcam.

“PD-Li” refers to a particular ligand of PD-1. Non-limiting exemplary amino acid sequences of PD-L1 can be found in the Uniprot database under accession numbers Q9NZQ7 (human PD-L1), Q9EP73 (murine PD-L1), and Q4QTK1 (pig PD-L1), other homologs of the same may also be found in the Uniprot database, i.e., at www.uniprot.org. Detection of a cell expressing PD-L1 can be identified using conventional techniques, such as the use of an anti-PD-L1 antibody, which are commercially available and approved for clinical use, e.g. from a vendor such as AstraZeneca (Imfinisi or drevalumab, Drug Bank Accession No. DB11714), EMD Serono (Avelumab, Drug Bank Accession No. DB11945), and Roche (Tecentriq or atezolizumab, Drug Bank Accession No. DB11595).

As used herein, “CTLA-4” or “cytotoxic T-lymphocyte-associate protein 4” or “CD152” is a protein that functions as an immune checkpoint to downregulate immune responses and is constitutively expressed in regulator T cells and upregulated in conventional T cells after activation—particularly in cancers. Non-limiting exemplary amino acid sequences for CTLA-4 can be found in the Uniprot database under accession numbers P16410 (human CTLA-4) and P09793 (murine CTLA-4); other homologs of the same may also be found in the Uniprot database, i.e., at www.uniprot.org. Detection of a cell expressing CTLA-4 can be identified using conventional techniques, such as the use of an anti-CTLA-4 antibody, which are commercially available, e.g., from a vendor such as BioLegend.

As used herein, “LAG3” or “lymphocyte-activation gene 3” or “CD223” or “cluster of differentiation 223” is a protein that is encoded by the LAG3 gene and belongs to the immunoglobulin (Ig) superfamily. LAG3 is a cell surface protein that is expressed in a variety of cell types, including T-cells, natural killer cells, B cells, and plasmacytoid dendritic cells. Non-limiting exemplary amino acid sequences for LAG3 can be found in the Uniprot database under accession numbers P18627 (human LAG3) and Q61790 (murine LAG3); other homologs of the same may also be found in the Uniprot database, i.e., at www.uniprot.org. Detection of a cell expressing LAG3 can be identified using conventional techniques, such as the use of an anti-LAG3 antibody, which are commercially available, e.g., from a vendor such as BioLegend.

As used herein, “TIM-3” or “HAVCR2” or “T-cell immunoglobulin and mucin-domain containing-3” is a protein known to be expressed on the surface of interferon gamma producing CD4+ Th1 cells, CD8+ Tc1 cells, Th17 cells, T regs, and innate immune cells TIM-3 is a known immune checkpoint protein. Non-limiting exemplary amino acid sequences for TIM-3 can be found in the Uniprot database under accession numbers Q8TDQ0 (human TIM-3) and Q8VIM0 (murine TIM-3); other homologs of the same may also be found in the Uniprot database, i.e., at www.uniprot.org. Detection of a cell expressing TIM-3 can be identified using conventional techniques, such as the use of an anti-TIM-3 antibody, which are commercially available, e.g., from a vendor such as ThermoFisher.

As used herein, “VISTA” or “V-domain Ig suppressor of T cell activation” is a type 1 transmembrane protein that functions as an immune checkpoint that is a member of the B7 family, primarily expressed in white blood cells. It can act as both a ligand and receptor on T cells to inhibit effector function and maintain peripheral tolerance. Non-limiting exemplary amino acid sequences for VISTA can be found in the Uniprot database under accession numbers Q9H7M9 (human VISTA) and Q9D659 (murine VISTA); other homologs of the same may also be found in the Uniprot database, i.e., at www.uniprot.org. Detection of a cell expressing VISTA can be identified using conventional techniques, such as the use of an anti-VISTA antibody, which are commercially available, e.g., from a vendor such as ThermoFisher.

In one aspect, the antibody is a monoclonal antibody. Monoclonal antibodies may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor, et al., Immunol. Today 4: 72 (1983)) and the EBV hybridoma technique to produce human monoclonal antibodies (see, e.g., Cole, et al., in: Monoclonal Antibodies in Cancer Therapy, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80: 2026-2030 (1983)) or by transforming human B-cells with Epstein Barr Virus in vitro (see, e.g., Cole, et al. (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the polypeptide. Alternatively, hybridomas expressing the monoclonal antibodies can be prepared by immunizing a subject, e.g., with an isolated polypeptide comprising, or alternatively consisting essentially of, or yet further consisting of, the amino acid sequence or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies and T-Cell Hybridomas, 563-681 (1981).

As noted above, the antibodies of the present disclosure are known in the art or can be produced through the application of recombinant DNA and phage display technology. For example, antibodies can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fv or disulfide stabilized Fv antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity for a polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)2 fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFvs) comprise a heavy chain variable region and a light chain variable region connected with a linker peptide (typically around 5 to 25 amino acids in length). In the scFv, the variable regions of the heavy chain and the light chain may be derived from the same antibody or different antibodies. scFvs may be synthesized using recombinant techniques, for example by expression of a vector encoding the scFv in a host organism such as E. coli. DNA encoding scFv can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFv and a host transformed by the expression vector can be obtained according to conventional methods known in the art.

Antigen binding fragments may also be generated, for example the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (Huse et al., Science, 256: 1275-1281 (1989)).

The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFv molecules, binding to streptavidin, introduction of a helix-turn-helix motif and the like can be exemplified.

The compositions disclosed herein may be in the form of a conjugate formed between any of these antibodies and another agent (such as an antibody drug conjugate (ADC) or an immunoconjugate). Non-limiting examples suitable to conjugate or fuse with the immunoconjugates disclosed herein include. detectable labels such as radioactive labels, an immunomodulator, a hormone, an enzyme, an oligonucleotide, a photoactive therapeutic or diagnostic agent, a cytotoxic agent, a drug or a toxin, an ultrasound enhancing agent, a non-radioactive label, a combination thereof and other such agents known in the art. In some embodiments, the antibodies may be conjugated to therapeutic agents, prodrugs, peptides, proteins, enzymes, viruses, lipids, biological response modifiers, pharmaceutical agents, and/or polyethylene glycol (PEG).

The oligonucleotides of the immunocongugate comprise an immunostimulatory sequence motif which contains at least one unmethylated CG dinucleotide and have in vivo immunostimulatory activity may be used to prepare the conjugates. In some embodiments, the oligonucleotide may be chemically modified to enable linkage to the antibody. Modification may involve adding a thiol group to the 3′ terminal nucleotide using a non-nucleoside linker (3′-thiol-modifier C3) (Zukermann et al., Nucleic Acids Res, 15: 5305-5321, 1987) to facilitate covalent linkage with linker modified antibody. The following CpG immunostimulatory oligonucleotides are exemplary (CpG motifs identified by bolded text with underlining).

| SEQ ID NO: 1 | |
|---|---|
| (CpG-1826): | 5′-TCCATGA [symbol] TTCCTGA [symbol] TT-3′ |
| | (class A) |
| SEQ ID NO: 2: | |
| (untitled): | 5′-TCTCCCAG [symbol] TGCGCCAT-3′ |
| | (class A) |
| SEQ ID NO: 3 | |
| (CpG-2395): | 5′-T [symbol] T [symbol] TTTT [symbol] G [symbol] C [symbol] |
| | (class C) |
| SEQ ID NO: 4 | |
| (CpG-1668): | 5′-TCCATGA [symbol] TTCCTGATGCT-3′ |

CpG immunostimulatory oligonucleotides for human application:

SEQ ID NO: 5

| | |
|---|---|
| (CpG-2006): | 5′-T [symbol] T [symbol] TTTTGT [symbol] TTTTGT [symbol] TT |
| | (class B) |

SEQ ID NO: 16

| | |
|---|---|
| (CpG 1585): | 5′ GGGGTCAACGTTGAGGGGGG 3′ |

SEQ ID NO: 17

| | |
|---|---|
| (CpG 2216): | 5′ GGGGGACGATCGTCGGGGGG 3′ |

SEQ ID NO: 18

| | |
|---|---|
| (CpG 2395): | 5′ TCGTCGTTTTCGGCGCGCGCCG 3′ |

SEQ ID NO: 9

| | |
|---|---|
| (CpG 5397): | 5′ TCGTCGTTTTCCGGCGCGCCGG 3′ |

SEQ ID NO: 10

| | |
|---|---|
| (CpG 2429): | 5′ TCGTCGTTTTCGGCGGCCGCCG 3′ |

SEQ ID NO: 11

| | |
|---|---|
| (K23): | 5′ TCGAGCGTTCTC 3′ |

SEQ ID NO: 12

| | |
|---|---|
| (D35): | 5′ GGTGCATCGATGCAGGGGGG 3′ |

SEQ ID NO: 13

| | |
|---|---|
| (CpG 2059): | 5′ TCGTCGTTTTGTCGTTTTCTCGT 3′ |

CpG immunostimulatory oligonucleotides having applications for human use include class A, B or C type CpG ODNs which are well known and may linked to a cancer targeting molecule as described herein. Exemplary such CpG immunostimulatory oligonucleotides are described in the following:

CpG 7909 for lymphoma therapy
Wooldridge, J, Link, B K, Weisdorf, D J, et al. Phase I study of oligodeoxynucleotide CpG 7909 in patients with previously treated non-Hodgkin's lymphoma. ASCO 2003; abstract 843.

CpG 2080
Hartmann, G. and Krieg, A M. Mechanism and function of a newly identified CpG DNA motif in human primary B cells. J. Immunol. 164:944-952, 2000.

K23 and D-35 ODN
Gursel, M, Verthelyi, D, Gursel, I, Ishii, K, and Klinman, D M. Differential and completive activation of human immune cells by distinct classes of CpG oligodeoxynucleotide. J. Leukocyte Biology 71:813-820, 2002.

Human Toll-like receptor 9 is optimally triggered by the motif GTCGTT
Bauer, S. et al. Human TLR9 confers responsiveness to bacterial DNA via species-specific CpG motif recognition. PNAS 98:9237-9242, 2001.
Hartmann et al. Delineation of a CpG phosphorothioate oligodeoxynucleotide for activating primate immune responses in vitro and in vivo. J. Immunology 164:1617-1624, 2000.

K3, K19, K110 and others (sequences shown)
Leifer, CA, Daniela, V, and Klinman, DM. Heterogeneity in the human response to immunostimulatory CpG Oligodeoxynucleotides. J. Immunotherapy 26:313-319, 2003.

CpG 2006 and C-2395
Vollmer et al. Characterization of three CpG oligodeoxynucleotide classes with distinct immunostimulatory activities. Eur. J. Immunol. 34:251-262, 2004.

CpG 2006
Gantner, F, Hermann, P, Nakashima, K, Matsukawa, S, Sakai, K, and Bacon, KB, CD40-dependent and -independent activation of human tonsil B cells by CpG oligodeoxynucleotides. Eur. J. Immunology 33:1576-1585, 2003.

CpG-A type (CpG 2216, CpG 1585); CpG-B (CpG 2006)
Rothenfusser, et al. CpG-A and CpG-B oligonucleotides differentially enhance human peptide-specific primary and memory CD8+ T-cell responses in vitro. Blood 103:2162-2169, 2004.

An exemplary CpG immunostimulatory oligonucleotide class A is CpG-1826 (Ballas et al., J. Immunol. 167: 4878-86, 2001), which has two motifs (5′-GACGTT-3′) and has been shown to induce immunostimulatory activity in mice (Baines et al., Clin. Cancer Res. (2003) 9:2693-2700; Lonsdorf et al. J. Immunol. (2003) 171:3941-3946). A 20-mer CpG ODN (SEQ ID NO: 2) is also useful because it has a significant effect on murine NK cells with little effect on murine B cells (Wooldridge et al., Blood (1997) 89:2994-2998). Other CpG ODN have been reported in the literature and can be used to link to an antibody or antigen binding fragment thereof (Krieg et al., Nature (1995) 374:546-549; Bauer et al., J. Immunol. (2001) 166:5000-5007).

SEQ ID NO: 3 has been described to be active on murine B-cells by Gursel et al. (J. Leukocyte Biol. (71:813-820), while a class C CpG motif, SEQ ID NO:4 (CpG-2395) was described by Vollmer et al. (Eur. J. Immunol. (2004) 34:252-262).

Particular oligonucleotides including the GpC type may be used as a negative control in experimental analysis of CpG immunostimulatory oligonucleotide and conjugates.

| SEQ ID NO: 6 | |
|---|---|
| (1745): | 5′-TCCAATGAGCTTCCTGAGTCT-3′ |
| | (negative control) |
| SEQ ID NO: 7 | |
| (GpC-1982): | 5′-TCCAGGACTTCTCTCA [symbol] TT-3′ |
| | (negative control) |
| SEQ ID NO: 8 | |
| (GpC-1668): | 5′-TCCATGAGGTTCCTGATGCT-3′ |
| | (negative control) |

SEQ ID NO: 6, (CpG-1745) has been previously shown to have no immunostimulatory activity.

CpG immunostimulatory oligonucleotides (or control sequences) may be synthesized by replacing the phosphodiester backbone with a phosphorothioate linkage (“PS linkage”). PS forms of CpG immunostimulatory oligonucleotides display an extremely high degree of nuclease resistance and stability (Stein et al. Nucleic Acids Res. (1988) 16:3209-3221). CpG immunostimulatory oligonucleotides also may be used in which part has the phosphodiester backbone and part has an alternative backbone such as a phosphorothioate linkage. CpG immunostimulatory oligonucleotide sequences not disclosed herein may be prepared along principles of those currently known. CpG immunostimulatory oligonucleotides may be prepared with different backbone chemistry provided that the resulting CpG immunostimulatory oligonucleotides can stimulate the immune response as described herein.

For quality assurance, endotoxin levels of all oligonucleotides, antibodies and the conjugates can be measured by Limulus amebocyte lysate assay (Bio-Whitaker, Walkersville, MD) to confirm that levels are below 0.01 Units/ml.

In one aspect the checkpoint inhibitor an anti-PD-L1 antibody or fragment thereof such as atezolizumab and the CpG immunostimulatory ODN is a TLR9 ODN wherein all bases of the sequence have the phosphorothioate backbone and the last base is substituted with a 3-′thiomodifier C3. In another aspect, the modified CpG is linked to the primary amines on the antibody or fragment thereof. In a further aspect, the checkpoint inhibitor an anti-PD-L1 antibody or fragment thereof such as atezolizumab and the immunostimulatory CpG comprises CpG 1826 (ODN 1826), and optionally wherein all bases of the CpG ODN have the phosphorothioate backbone and the last base is substituted with a 3-′thiomodifier C3. In another aspect, the checkpoint inhibitor an anti-PD-L1 antibody or fragment thereof such as atezolizumab and the immunostimulatory CpG comprises CpG 2006, and optionally wherein all bases of the CpG 2006 have the phosphorothioate backbone and the last base is substituted with a 3-′thiomodifier C3. In another aspect, the modified CpG is linked to the primary amines on the antibody or antigen binding fragment thereof.

Further provided herein are polynucleotides encoding the polypeptide backbones of the immunoconjugates, vectors comprising the polynucleotides (optionally with regulatory and enhancer elements) and host cells containing them. The polynucleotides can be operatively linked to regulatory sequences such as promoters and/or enhancers for replication or expression. The polynucleotides can be contained within expression or replication vectors and used in host cell systems for expression or replication thereof. Host cells are described above. Thus, in another aspect, provided herein is a method to replicate or express the polynucleotide by culturing a host cell containing the polynucleotide with the appropriate regulatory sequences under conditions that promote expression and/or replication. Further provided is isolation or purification of the expression or replication products using methods known in the art.

### Compositions

Additional aspects of the disclosure relate to compositions comprising a carrier and one or more of the immunoconjugates, polynucleotides, and/or host cells as described herein and optionally a carrier such as a pharmaceutically acceptable carrier.

Briefly, pharmaceutical compositions of the present disclosure including but not limited to any one of the disclosed compositions, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration. In addition, preservatives can be added to the compositions.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

The compositions can further comprise an effective amount of another therapy, such as an immunotherapy or chemotherapy. In one aspect the compositions further comprise an HDAC inhibitor such as Entinostat. In one aspect, the compositions comprise an immunoconjugate comprising ODN 2006 or ODN 1826 as described herein conjugated to an anti-PD-L1 antibody or antigen binding fragment thereof (e.g., Tecentriq) and Entinostat.

### Methods

Also provided herein is a method to inhibit the growth of a cancer cell or a tumor cell. In one aspect the cancer cell or tumor expresses an immune checkpoint. In one aspect, the immune checkpoint is of the group of PD-L1, CTLA-4, and LAG3, TIM-3, or VISTA. In a particular aspect, the immune checkpoint is PL-L1.

In another aspect the cancer or tumor cell expresses the immune checkpoint and is resistant to immune checkpoint therapy. The cell can be a cultured cell or primary cell from a biopsy. Cultured cells are commercially available. The cancer or tumor cell can be a solid tumor cell, e.g., a breast cancer cell, a colon cancer cell or a melanoma. In a further aspect, the breast cancer cell is a triple negative breast cancer cell. In a further aspect, the cell or tumor expresses an immune checkpoint such as PD-L1. In one aspect, the cell is selected from D2F2, 4T1, B16 or C26. In a further aspect, it expresses the immune checkpoint but is resistant to the appropriate immune checkpoint inhibitor therapy, e.g., an anti-PD-L1 therapy to treat a cell or tumor expressing PD-L1.

The method comprises or consists essentially of, or yet further consists of contacting the cancer cell or tumor cell with an effective amount of the immunoconjugate and/or a composition containing the immunoconjugate. In one aspect, the immunoconjugate is matched to the checkpoint expressed by the cancer or tumor cell, e.g., an anti-PD-L1 antibody to a cancer or tumor expressing PD-L1.

In one aspect the cancer cell or tumor expresses PD-L1 and is optionally resistant to PD-L1 checkpoint inhibitor therapy, and the immunoconjugate comprises an anti-PD-L1 antibody or fragment thereof such as atezolizumab and the CpG immunostimulatory ODN is a TLR9 ODN wherein all bases of the sequence have the phosphorothioate backbone and the last base is substituted with a 3-′thiomodifier C3. In another aspect, the modified CpG is linked to the primary amines on the anti-PD-L1 antibody or fragment thereof. In a further aspect, the checkpoint inhibitor an anti-PD-L1 antibody or fragment thereof such as atezolizumab and the immunostimulatory CpG comprises, consists essentially of or yet further consists of CpG 1826, and optionally wherein all bases of the CpG ODN have the phosphorothioate backbone and the last base is substituted with a 3-′thiomodifier C3. In another aspect, the checkpoint inhibitor an anti-PD-L1 antibody or fragment thereof such as atezolizumab and the immunostimulatory CpG comprises CpG 2006, and optionally wherein all bases of the CpG 2006 have the phosphorothioate backbone and the last base is substituted with a 3-′thiomodifier C3. In another aspect, the modified CpG is linked to the primary amines on the antibody or fragment thereof. In a yet further embodiment, the cell or tumor is simultaneously or separately contacted with an HDAC inhibitor such as Entinostat.

The cancer cell or tumor cells can be an animal such as a mammal, e.g., a canine cell, a feline cell, or a human cell. The cell or tumor can be contacted with another anti-tumor or anti-cancer therapy. The contacting can be in vitro or in vivo. When contacted in vitro, the method provides a screen or assay for new therapies or for pre-screening personalized therapy. When contacted in vivo in a non-human animal, the method provides an animal model to test new therapies.

The compositions are intended for use in cancer therapy to treat, reduce the size, tumor burden or metastatic potential of the tumor or to induce an immune response in the subject having the cancer or tumor. Thus, provided herein is a method of treating a tumor or cancer comprising, or alternatively consisting essentially of, or yet further consisting of, administering an effective amount of the immunoconjugate or a composition containing the immunoconjugate to a subject in need thereof. Also provided is a method of inducing or raising an immune response in a subject having a tumor or cancer, the method comprising, or alternatively consisting essentially of, or yet further consisting of, administering an effective amount of the immunoconjugate or a composition containing the immunoconjugate to a subject in need thereof. In one aspect the cancer or tumor expresses an immune checkpoint. In one aspect, the immune checkpoint is of the group of PD-L1, CTLA-4, and LAG3, TIM-3, or VISTA. In a particular aspect, the immune checkpoint is PL-L1. In another aspect the cancer or tumor cell expresses the immune checkpoint and is resistant to immune checkpoint therapy. In one aspect, the immunoconjugate is matched to the checkpoint expressed by the cancer or tumor cell, e.g., an anti-PD-L1 antibody to a cancer or tumor expressing PD-L1.

In one aspect the cancer cell or tumor expresses PD-L1 and is optionally resistant to PD-L1 checkpoint inhibitor therapy, and the immunoconjugate comprises an anti-PD-L1 antibody or fragment thereof such as atezolizumab and the CpG immunostimulatory ODN is a TLR9 ODN wherein all bases of the sequence have the phosphorothioate backbone and the last base is substituted with a 3-′thiomodifier C3. In another aspect, the modified CpG is linked to the primary amines on the anti-PD-L1 antibody or fragment thereof. In a further aspect, the checkpoint inhibitor an anti-PD-L1 antibody or fragment thereof such as atezolizumab and the immunostimulatory CpG comprises CpG 1826, and optionally wherein all bases of the CpG ODN have the phosphorothioate backbone and the last base is substituted with a 3-′thiomodifier C3. In another aspect, the checkpoint inhibitor an anti-PD-L1 antibody or fragment thereof such as atezolizumab and the immunostimulatory CpG comprises CpG 2006, and optionally wherein all bases of the CpG 2006 have the phosphorothioate backbone and the last base is substituted with a 3-′thiomodifier C3. In another aspect, the modified CpG is linked to the primary amines on the antibody or fragment thereof. In a yet further embodiment, the cell or tumor is simultaneously or separately contacted with an HDAC inhibitor such as Entinostat. In one aspect, the cancer or tumor is a PD-L1 expressing breast cancer cell.

The cancer or tumor cell can be a solid tumor cell, e.g., a breast cancer cell, a colon cancer cell or a melanoma. In a further aspect, the breast cancer cell is a triple negative breast cancer cell. It can be a primary tumor or a metastatic tumor.

The subject can be an animal in need of therapy, such as a canine, a feline, or a human patient. The therapy can be combined with another anti-tumor or anti-cancer therapy by administering an effective amount of an anti-tumor or anti-cancer therapy, examples of which are described above. They can be administered concurrently or subsequent or serially. Administration can be determined by the treating physician or veterinarian, and can be locally or systemic, e.g., by i.p. injection. Dosing can be in one or more doses. The immunoconjugate can be administered as a first line, a second line, a third line, or a fourth line therapy. In one aspect, the immunoconjugate is administered subsequent to cytoreductive therapy.

The therapeutic methods can be combined with a diagnostic to determine the subject in need of such treatment who may best respond to it. Using PD-1 as an example, a sample is isolated from the patient to determine if the sample contains PDL-1. If the sample does, the patient is suitably treated by the methods. Thus, the therapeutics as described herein can be combined with therapeutics prior to and during treatment, and the therapeutic can be provided to the subject having the correct genotype or phenotype for that therapy.

The immunoconjugate can be combined with, or adjunctive to, one or more other treatments. Other treatments include, without limitation, chemotherapeutic treatment, antibody therapy, viral delivery potentiators, radiation therapy, surgical resection, imaging or ultrasound-guided delivery, etc.

Combination therapy as provided herein involves the administration of at least two agents to a patient, the first of which is an immunoconjugate, and the second is another therapeutic agent, wherein the first and the second therapeutic agents can be administered simultaneously, successively, or separately.

As used herein, the immunoconjugate and the other therapeutic agent are said to be administered successively if they are administered to the patient on the same day, for example during the same patient visit. Successive administration can occur 1, 2, 3, 4, 5, 6, 7 or 8 hours apart. In contrast, the combination of the disclosure and the other therapeutic agent are said to be administered separately if they are administered to the patient on the different days, for example, the combination of the disclosure and the other therapeutic agent can be administered at a 1-day, 2-day or 3-day, one-week, 2-week or monthly intervals. In the methods of the present disclosure, administration of the combination of the disclosure can precede or follow administration of the other therapeutic agent.

As a non-limiting example, the instant combination and other therapeutic agent can be administered concurrently for a period of time, followed by a second period of time in which the administration of the immunoconjugate and the other therapeutic agent is alternated.

Combination therapies of the present disclosure can result in a greater than additive, or a synergistic, effect, providing therapeutic benefits where neither of the CpG or checkpoint inhibitor nor other therapeutic agent is administered in an amount that is, alone, therapeutically effective. Thus, such agents can be administered in lower amounts, reducing the possibility and/or severity of adverse effects.

### Kits

Aspects of this disclosure relate to kits comprising the immunoconjugate and instructions for use. In one aspect the immunoconjugate comprises an anti-PD-L1 antibody or fragment thereof such as atezolizumab and the CpG immunostimulatory ODN is a TLR9 ODN wherein all bases of the sequence have the phosphorothioate backbone and the last base is substituted with a 3-′thiomodifier C3. In another aspect, the modified CpG is linked to the primary amines on the anti-PD-L1 antibody or fragment thereof. In a further aspect, the checkpoint inhibitor an anti-PD-L1 antibody or fragment thereof such as atezolizumab and the immunostimulatory CpG comprises CpG 1826, e.g. comprising TCC ATG ACG TTC CTG ACG TT (SEQ ID NO: 1) and optionally wherein all bases of the CpG ODN have the phosphorothioate backbone and the last base is substituted with a 3-′thiomodifier C3. In another aspect, the checkpoint inhibitor an anti-PD-L1 antibody or fragment thereof such as atezolizumab and the immunostimulatory CpG comprises CpG 2006, and optionally wherein all bases of the CpG 2006 have the phosphorothioate backbone and the last base is substituted with a 3-′thiomodifier C3. In another aspect, the modified CpG is linked to the primary amines on the antibody or fragment thereof. In a further aspect, the kit also comprises an HDAC inhibitor such as Entinostat.

In further embodiments, the instructions recite the method steps disclosed herein. The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit. As amenable, these suggested kit components may be packaged in a manner customary for use by those of skill in the art. For example, these suggested kit components may be provided in solution or as a liquid dispersion or the like. In a further aspect, reagents and instructions for a companion diagnostic is provided in the kit and/or the instructions for performing the method can be provided on the label or in the kit.

The following examples are provided to illustrate, and not limit the disclosure.

### EXAMPLES

### Example 1—Generation of CpG-PD-L1 Conjugates

In the last several years, the implementation of checkpoint inhibitor antibodies into clinical practice for the treatment of solid tumors has spurred the interest of oncologists to identify other treatments that can work synergistically with this new class of therapeutics. From these studies, it has been shown that innate immune agonists such as TLR-4 (oncolytic viruses), the Sting pathway, and TLR-9 (CpG) are perhaps the most potent enhancers of checkpoint inhibition associated with the PD-1/PD-L1 pathway. Without being bound by theory, and as an non-limiting example only, an antibody or antigen binding fragment thereof directed to PD-L1 conjugated to CpG would take advantage of this synergy and produce a biobetter checkpoint inhibitor. PD-L1 has the added advantage of being expressed on the surface of tumor cells as well as antigen presenting cells, the very cells that harbor the receptors for TLR-9. Without being bound by theory, conjugation of CpG with αPD-L1 may alter the toxicity profile of the checkpoint inhibitor in a favorable manner making thereby improving its use in the clinic.

Animal and Tumor Models: The mouse mammary tumor cell line, D2F2, was purchased from the American Type Culture Collection (ATCC). Cells were handled according to the protocols of the supplier and kept in culture no longer than 20 passages. Authentication of the cell line including check of post free viability, growth properties and morphology, test for mycoplasma contamination, isozyme assay and sterility were performed by the cell bank before shipment. Six-week-old female BALB/cJ mice were purchased from Jackson Laboratories (Bar Harbor, ME) and were maintained in accordance with IACUC approved institutional guidelines and protocols.

Antibody Immunoconjugate (AIC) Conjugation: The chemical conjugation of thiol-modified CpG to primary amines on antibodies has been previously described (Li et al. J Immunol Methods 389:45-51 (2013)). Briefly, antibodies were incubated with Sulfo-EMCS (Pierce, Rockford, IL) at 1:6 antibody to Sulfo-EMCS ratio in PBS containing 1 mM EDTA with continuous rocking for 1 hour at room temperature. The excess cross-linker was removed using Zeba™ Spin Desalting Columns (Pierce, Rockford, IL). Thiol-modified CpG1826 was reduced in 0.1 M DTT for 1 hour at room temperature. Excess DTT was then removed using Zeba™ Spin Desalting Columns. Reduced thiol-modified CpG was mixed with antibody/EMCS at a 6:1 ratio overnight at 4° C. Unconjugated CpG was separated from CpG conjugated CpG by gel filtration using a Sephacryl S-100 column (GE Healthcare, Little Chalfont, United Kingdom). For these studies, the clinically approved αPD-L1 antibody, Tecentriq (atezolizumab, Roche) was used since like other αhuman PD-L1 antibodies, these reagents are also effective cross-species including mice.

AIC Therapy Studies: Exponentially growing D2F2 cells were harvested using Detachin, washed twice in PBS, and mixed in a 50:50 solution with Matrigel. The right flank of mice were then inoculated by subcutaneous (s.c) injection with 5×10⁶ D2F2 cells/mouse in 200 uL of slurry. After 11 days, all mice presenting with palpable tumors were randomized into groups (n=5) and were subsequently treated with intraperitoneal (i.p.) administration of single agent therapies, CpG-B (1826) 20 μg/mouse or αPD-L1 200 μg/mouse, combined therapy of CpG+αPD-L1 (20 μg/mouse+200 μg/mouse), or targeted AIC therapy CpG/αPD-L1 220 μg/mouse (Table 2). Doses were delivered in 100 uL of PBS once a day (q.d.) for 5 days in a row. CpG Caliper measurements (mm) of tumor diameters (long, D and short, d) were taken three times a week and volumes were calculated as D/2*d² (mm³). Some mice were prematurely sacrificed as a result of severe ulcerating tumors. Remaining mice were followed until tumors reached end points of 1.5 cm in diameter or 1,500 mm³ in volume or until 18 days post-randomization and were then euthanized.

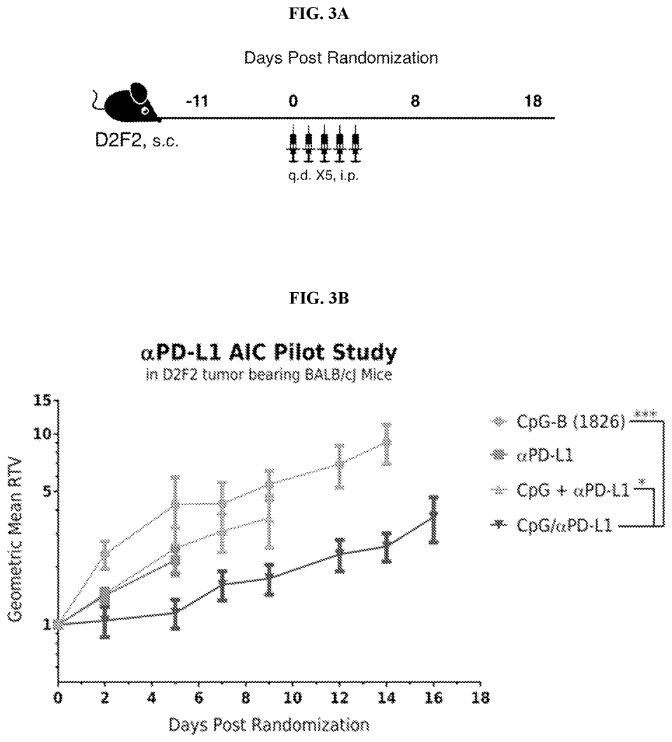

Statistical Analysis: As shown below in FIG. 3A-B, tumor volume data were analyzed according to guidelines published by AstroZeneca (Shaw et al. Lab Animal, 46(5), 207-211 (2017)). Relative Tumor Volumes (RTV) were calculated using measurements taken on day, n post-randomization (TVₙ) and day of randomization (TV₀), RTVn=TVn/TV₀. These data were log₁₀ transformed and then analyzed. Statistical analysis was performed using 2-tailed unpaired Student's t tests (when comparing 2 groups) or 1-way ANOVA (when comparing >2 groups) using Prism 5.0 (GraphPad) software. P values of less than 0.05 were considered statistically significant. Percent decrease (x %) of treated group relative to control group were calculated as x %=(1−RTV(T)/RTV(C))*100.

TABLE 2
Groups used to study the effect of combination CpG versus
conjugated CpG with αPD-L1 checkpoint inhibition (abbreviations:
ip—intraperitoneal, q.d.—once per day).

| | | | (μg/mouse) | | |
|---|---|---|---|---|---|
| Group | | Therapy | q.d. X5 | Route | n |
| 1 | Single | CpG-B (1826) | 20 | i.p. | 5 |
| 2 | Single | αPD-L1 | 200 | i.p. | 5 |
| 3 | Combination | CpG + αPD-L1 | 20 + 200 | i.p. | 5 |
| 4 | AIC | CpG/αPD-L1 | 220 | i.p. | 5 |

| | |
|---|---|
| Total Mice (N) | 20 |

Results: The syngeneic model of immunocompetent BALB/cJ mice bearing subcutaneous tumor engraftment of D2F2 murine breast carcinoma was used to test the tumor growth inhibition activity of the CpG/αPD-L1 AIC in vivo. As shown below in FIGS. 3A-3B, while no single therapeutic group demonstrated a partial or complete response, the CpG/αPD-L1 AIC demonstrated a statistically significant delay in tumor growth compared to both free CpG-B (71.6% inhibition, p<0.001) and combination therapy CpG+αPD-L1 (51.8% inhibition, p=0.025). Interestingly, only the group treated with CpG/αPD-L1 AIC remained ulcer free.

Toll-like receptor 9 agonists consisting of the three classes of CpG motifs in the past have been used exclusively as free reagents injected directly into tumors or used as adjuvants for vaccine preparations. Prior work with Class B and C CpG ODN have shown that after intratumor injection in combination with vaccine immunotherapy, CpG can be effective in suppressing tumor growth. By contrast, Applicants have demonstrated, for the first time that these potent innate immune stimulators can be targeted to tumors after conjugation to antibodies directed against tumor components and can induce significant immunotherapy without the use of vaccine technologies. Since TLR-9 receptors are found only in the cytoplasm of antigen presenting cells, it was thought that antibody targeting of these reagents was not possible. Applicants' data showed, however, that if CpG was targeted to the tumor site, it is selectively taken up by antigen presenting cells and translocated into cytoplasmic endosomes where it bound TLR-9 receptors. By contrast, the antibody was left on the surface of the tumor at the site of antibody binding. Because of these surprising findings, conjugation of CpG to tumor targeting antibodies can now be administered systemically by intravenous injection circumventing the need to administer these rapidly clearing TLR-9 agonists locally. In prior disclosures, CpG was conjugated to antibodies that targeted necrotic regions of tumors and found to be very active in suppressing tumor growth. The present disclosure now describes the conjugation of CpG to checkpoint inhibitor antibodies such as αPD-L1 and shows that after conjugation, these drug conjugates retain their activity and enhance the therapeutic effects of checkpoint inhibition to generate a “biobetter” αPD-L1 product. To demonstrate this finding, the combination of free CpG with αPD-L1 had 71% less inhibition than the CpG antibody conjugate at day 14 after the initiation of therapy. In addition, it was observed that tumor-bearing mice treated with conjugated CpG showed no evidence of scabbing on the tumor surface compared to other groups that received unconjugated PD-L2. These data can be interpreted to indicate that after conjugation with CpG, αPD-L1 may be less toxic which is a very desirable characteristic for clinical use. In summary, Applicants have demonstrated in in vivo experiments that targeting a potent CpG agonist via conjugation to a checkpoint inhibitor antibody such as αPD-L1, can significantly engage innate immunity at the tumor site to provide a more effective method of immunotherapy of difficult to treat solid tumors.

### Example 2—T1 CPG/PD-L1

A second murine breast carcinoma model, 4T1, which is a triple negative tumor and PD-L1 resistant was used to test the clinical effectiveness of CpG/PD-L1 immunoconjugate (AIC) on tumor growth and mouse survival. This mouse tumor was selected since it is also highly metastatic in immunocompetent mice and is therefore a rigorous tumor model for these experiments.

Material and Methods: Eight-week-old BALB/c female mice purchased from Jackson Laboratories (Bar Harbor, ME) were injected in the right mammary fat pad using 5×10⁴ cells in 0.1 inoculum in sterile Phosphate Buffered Saline. Mice were randomized into six groups (n=6) and injected for 5 consecutive days as follows:

Group 1. Vehicle alone control (25 mM Histidine, 3.75% sucrose, 0.04% tween which is the AIC storage buffer given ip and 0.5% methyl cellulose administered orally (vehicle for Entinostat)

Group 2. AIC+Entinostat. Six doses of Entinostat were administered by oral gavage at a dose of 5 mg/kg Entinostat (Syndax, Inc.) suspended in 0.5% methyl cellulose one day before administration of AIC. CpG/PD-L1 (AIC) was administered at a dose of 250 ug in 100 ul inoculum injected ip. for five consecutive days.

Group 3. AIC alone. Treated as above.

Group 4. PD-L1+Entinostat. Five doses of Entinostat were given as above and the PD-L1 (Tecentriq, Genentech) was administered twice a week for one week at a dose of 200 ug/dose in a 100 ul inoculin ip.

Group 5. PD-L1 alone control (same as group 4).

Group 6. CpG+PD-L1 control. Free CpG (50 ug) mixed with PD-L1 (200 ug) was administered daily for 5 consecutive days ip.

Mice were monitored 3×/week using a digital caliper for tumor size and tumor volume was calculated by the formula length×width²/2. From these data, a Kaplan Meier Plot was derived to demonstrate the effects of treatment on mouse survival. Tumor growth curves were also derived from the data for each mouse.

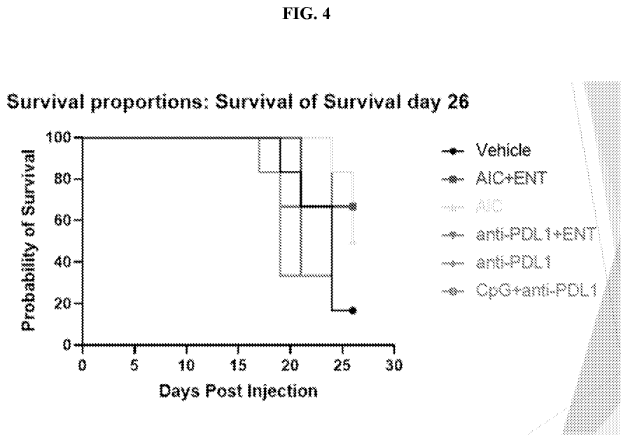

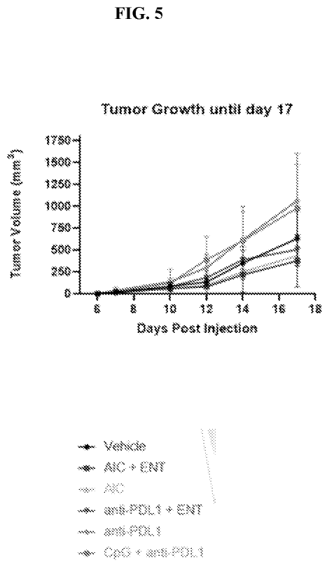

Results: Tecentriq anti-PD-L1, a commercially approved humanized PD-L1 chemically linked to the Toll-Like receptor 9 agonist CpG was used and compared to untreated, PD-L1 alone, and also added two new groups to see if the HDAC inhibitor Entinostat improves the CpG/PD-L1 antibody immunoconjugate (AIC). Like the first experiment, the CpG/PD-L1 was effective in the survival data as shown by the Kaplan Meier Plot in this second breast cancer tumor model. See FIG. 4 and FIG. 5.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

Murad and Clay. “CpG Oligonucleotides at TLR9 Agonists.” Biodrugs, 23(6):361-375 (2009).
Duraiswamy et al. “Therapeutic PD-1 Pathway Blockade Augments with Other Modalities of Immunotherapy T-Cell Function to Prevent Immune Decline in Ovarian Cancer.” Cancer Res. 73(23):6900-6912 (2013).
Lines et al. “VISTA Is an Immune Checkpoint Molecule for Human T Cell.” Cancer Res. 74(7):1924-1932 (2014).
Guo et al. “PD-1 Blockade and OX40 Triggering Synergistically Protects against Tumor Growth in Murine Model of Ovarian Cancer.” PLOS One 9(2):e89350 (2014).
Chiba et al. “Tumor-infiltrating DCs suppress nucleic acit-mediated innate immune responses through interactions between the receptor TIM-3 and the alarmin HMGB1.” Nature Immunol. 13(9): 832-842 (2012).
Contardi et al. “CTLA-4 is constitutively expressed on tumor cells and can trigger apoptosis upon ligand interaction.” Int. J. Cancer. 117:538-550 (2005).

1. An immunoconjugate comprising an immune checkpoint inhibitor linked to an oligonucleotide comprising an immunostimulatory sequence motif which contains at least one unmethylated CG dinucleotide and optionally, a detectable or purification label.

2. The immunoconjugate of claim 1, wherein the immune checkpoint inhibitor comprises an antibody or an antigen binding fragment thereof of an immune checkpoint inhibitor.

3. The immunoconjugate of claim 2, wherein the antibody comprise an antibody selected from an anti-PD-L1 antibody, an anti-CTLA-4 antibody, an anti-LAG3 antibody, an anti-TIM-3 antibody, or an anti-VISTA antibody.

4. The immunoconjugate of claim 2, wherein the antigen binding fragment comprises the CDRs of an antibody selected from the group of an anti-PD-L1 antibody, an anti-CTLA-4 antibody, an anti-LAG3 antibody, an anti-TIM-3 antibody, or an anti-VISTA antibody.

5. (canceled)

6. The immunoconjugate of claim 1, wherein the immune checkpoint inhibitor comprises the CDRs of atezolizumab.

7. The immunoconjugate of claim 1, wherein the immunostimulatory sequence motif is CpG oligodeoxynucleotide (ODN), optionally a CpG Class-A ODN, a CpG Class-B ODN, a CpG Class-C ODN or the Toll-Like receptor 9 agonist CpG.

8. The immunoconjugate of claim 1, wherein the immunostimulatory sequence motif is thiol-modified and, optionally comprises CpG1826 or CpG 2006 and further optionally wherein all bases of the CpG have the phosphorothioate backbone and the last base is substituted with a 3′-thiomodifier C3.

9. The immunoconjugate of claim 1, wherein the immunostimulatory sequence motif comprises the Toll-Like receptor 9 agonist CpG.

10.-11. (canceled)

12. An isolated polynucleotide encoding an immune checkpoint inhibitor and an immunostimulatory sequence motif which contains at least one unmethylated CG dinucleotide and optionally, a detectable or purification label, optionally further comprising a regulatory polynucleotide.

13-17. (canceled)

18. A method for inhibiting the growth of a cancer cell or tumor, comprising contacting the cell or tumor with the immunoconjugate of claim 1, thereby inhibiting the growth of the cancer cell or tumor.

19.-24. (canceled)

25. A method of treating a tumor or cancer in a subject in need thereof, comprising administering an effective amount the immunoconjugate of claim 1, thereby treating the cancer or tumor.

26. A method of inducing an immune response in a cancer subject, comprising administering an effective amount the immunoconjugate of claim 1, thereby treating inducing the immune response in the subject.

27.-37. (canceled)

38. A method for preparing an immunoconjugate comprising chemically crosslinking an immune checkpoint inhibitor linked to an oligonucleotide comprising an immunostimulatory sequence motif which contains at least one unmethylated CG dinucleotide.

39.-44. (canceled)

45. A kit comprising the immunoconjugate of claim 1 and instructions for use.
